(19) United States
(12) Patent Application Publication
(10) Pub. No.: US 2007/0243217 A1
(43) Pub. Date: Oct. 18, 2007

(54) Film-Shaped Medicament for Oral Administration Containing Estriol

(75) Inventors: Joachim Moormann, Werne (DE); Walter Elger, Berlin (DE)

Correspondence Address: D. PETER HOCHBERG CO. L.P.A., 1940 EAST 6TH STREET, CLEVELAND, OH, 44114, US

(73) Assignee: HF Arzneimittelforschung GmbH, Werne (DE)

(21) Appl. No.: 11/596,239

(22) PCT Filed: May 6, 2005
(86) PCT No.: PCT/EP05/04894

§ 371 Date: Nov. 13, 2006

(30) Foreign Application Priority Data
May 14, 2004 (DE) 10 2004 023 984.3

(51) Int. Cl. A61K 9/00

(52) U.S. Cl. 424/400

(57) ABSTRACT

Film-shaped medicaments administered orally, particularly buccally, for treating climacteric complaints. The medicaments contain estriol and/or at least one pharmacologically acceptable ester of estriol, either alone or in combination with at least one gestagen.

### CROSS-REFERENCE TO RELATED APPLICATIONS

This application is a National Stage application of International Application No. PCT/EP2005/004894, filed on May 6, 2005, which claims priority of German application number 10 2004 023 984.3, filed on May 14, 2004.

### BACKGROUND OF THE INVENTION

1. Field of the Invention

The present invention relates to film-shaped medicaments which are to be administered orally and which are intended for transmucosal administration of estriol and/or at least one of its pharmacologically acceptable esters, alone or in combination with at least one gestagen.

The present invention further relates to the use of estriol and/or at least one of its pharmacologically acceptable esters, alone or in combination with at least one gestagen, for the production of a film-shaped, orally administered medicament for the treatment of climacteric complaints.

2. Description of the Prior Art

The menopause of women (climacteric) is a period of life separating the fertile phase from the period in which reproduction is no longer possible. The climacteric period is characterized by a continuous decrease in the hormone production of the ovaries as well as by a decrease in, and ultimately the cessation, of the menstrual period which is associated therewith. At the beginning of the climacteric period, the gestagen level in the blood decreases and after several years less estrogen is produced as well, until the body ceases to produce these hormones entirely.

During this phase of hormonal change, complaints occur in around 80% of women. The ebbing hormone production causes organic complaints. Among these are dry, thin and slackening skin, a reduction in muscular tension, altered hairiness or even loss of hair, arthralgia and myalgia, increased water deposition in the tissue, leading to tension in the breasts, a dry and itching vagina, more frequent inflammations of the vagina, inflammations of the bladder, and dribbling of urine, as well as a decrease in bone mass (osteoporosis).

Organic complaints may be accompanied by neurovegetative complaints such as hot flashes, outbreaks of sweat, disturbed sleep, vertigo, tachycardia or headaches. These symptoms determine the picture of the climacteric complaints for about 2 to 3 years.

Additionally, psychic complaints may occur. Among these are anxiety states, irritability and aggressiveness, inner tension, bad mood, nervousness, mood swings and depression, fatigue and lack of drive, forgetfulness, diminished concentration power, and altered sexuality. These complaints usually lessen again when the organism has grown used to the lack of hormones.

The complaints associated with the climacteric period are felt to be rather mild by 30 to 40% of the women affected, in 25% of those affected the psychic and somatic complaints are very strong, and 5% of the women concerned are even temporarily incapable of working.

In the so-called hormone replacement therapy (HRT), estrogens combined with gestagens are administered to compensate for the hormone deficit and to treat the complaints associated with the climacteric period.

The benefit of hormone replacement therapy for treating climacteric complaints in women in postmenopausal phase and for the prophylaxis of osteoporosis and cardiovascular diseases is well proven.

There is, however, also a possibility of unwanted side effects occurring in hormonal replacement therapy. Among the most frequent side effects are increased formation of oedemas, increased risk of thrombosis, increased risk of acquiring an endometrial carcinoma, feeling of tension in the breasts, gain in weight, nausea, hyperpigmentation and spotting.

In addition, a continual administration of hormonally active estrogens promotes the proliferation of vaginal epithelial cells and increases the risk of hyperplasia of the endometrium.

In view of the disadvantages associated with continual estradiol admininistration it has been proposed to use estriol, which scarcely induces proliferation of the endometrium, instead of estradiol within the framework of hormone replacement therapy. Estriol ((16α, 17β)-estra-1,3,5(10)-triene-3,16,17-triol) is the final metabolic product and the intermediary metabolic product, usually predominant in the urine, of the estrogen metabolism. Being a metabolite of estradiol, the hormonal effect of estriol is also substantially smaller than that of estradiol.

However, when administered perorally in the form of coated tablets, capsules or tablets, estriol is quickly inactivated in the liver by formation of its glucoronide derivative. Thus, only 1 to 2% of the administered estriol dose enters the blood circulation in bioavailable form. For this reason, for peroral administration of estriol a dose of 2 mg per day has been found optimal to prevent vaginal atrophy and effective in the treatment of hot flashes, disturbed sleep and several other problems associated with the climacteric period. Because of the relatively high dose (2 mg/day) administered, there is also a possibility of side effects occurring such as nausea and mastalgia.

As an alternative to peroral administration, the vaginal administration of estriol has also been tested in order to be able to reduce the amount of the hormone which has to be administered while retaining the same systemic effect. A smaller dose additionally has the advantage of a smaller deflection of estrogen-modulated liver functions. Although vaginal administration proved to be more effective than peroral administration since intravaginally administered estriol was initially quickly absorbed and is therefore in principle suitable for local as well as systemic hormone replacement therapy, the hornification of the vaginal epithelium which occurs in prolonged application and which has a negative effect on the absorption of estriol militates against the vaginal application of estriol. Moreover, the vaginal administration of estriol was not accepted by the probands.

Apart from a vaginal administration, in EP 0 630 248 it has been described that transdermally administered estriol is also suitable for the therapy of climacteric osteoporosis. However, the very high estriol amounts (12 mg per 24 hours) which had to be continuously administered transdermally to the male subjects in order to achieve a serum level of free estriol that corresponded to the physiological concentrations of estrogenic hormones in the female cycle (50 to 350 μg/ml) must be considered to be disadvantageous.

Apart from this, the transdermal administration of estriol should result in a largely constant blood level of estriol which, it is true, has a positive effect on the bones, but also promotes the unwanted effects on the mucosa of the uterus.

### SUMMARY OF THE INVENTION

It was therefore the object of the present invention to provide a medicament for administration of estriol which is to be administered orally and is suitable for the treatment of climacteric and/or postmenopausal complaints, and which avoids the aforementioned disadvantages of the known peroral administration forms as far as possible.

It has surprisingly been found that this object is achieved by a monolayer or multilayer film-shaped medicament containing estriol and/or at least one pharmacologically acceptable ester of estriol.

### DETAILED DESCRIPTION OF THE PRESENT INVENTION

Suitable pharmacologically acceptable esters of estriol are, for example, estriol triacetate, estriol tripropionate, estriol-3-acetate, estriol-16-acetate, estriol-16,17-diacetate, estriol-3-17-disulfate, estriol-16,17-disulfate, estriol-3-sulfate, estriol-17-sulfate, estriol-3-hemisuccinate or estriol-16,17-hemisuccinate.

Estriol and/or the pharmacologically acceptable ester(s) of estriol may optionally be contained in the film-shaped medicament in combination with at least one gestagen.

The film-shaped medicaments according to the invention enable the transmucosal absorption of estriol and/or of the pharmacologically acceptable ester(s) of estriol and, as the case may be, of the gestagen(s) additionally contained in the medicament, via the oral mucosa by applying the medicament sublingually or buccally. The active substance or the active substances contained in the film-shaped medicament is/are released from the film-shaped medicament as a result of the action of saliva, and subsequently can be absorbed via the oral mucosa. In this way, the so-called “first pass effect”, which is responsible for the quick inactivation of perorally administered estriol, is avoided. The dose of estriol and/or estriol ester, relative to the known administration forms for estriol, can thereby be reduced to less than 2 mg/24 hours, preferably to approximately 200 μg per 24 hours, released by the inventive medicament, in order to achieve the same systemic effects as with markedly higher doses of perorally or transdermally administered estriol.

Furthermore, no changes of the oral mucosa analogous to the proliferation and hornification of the upper cell layers in the vagina observed in the vaginal administration of estrogens were observed with buccal or sublingual administration of estriol.

These changes in the vagina might be the reason for the observed fact that, with the same treatment and dose, the estriol levels in the blood drop measurably after some time when estriol is applied vaginally, but not when it is administered transmucosally via the oral mucosa. Thus, even in the case of long-term use, there should be no unwanted absorption conditions at the oral mucosa as the application site that show a change for the worse, provided that the treatment of the climacteric complaints is carried out using a film-shaped medicament according to the invention.

The film-shaped administration forms according to the invention are medicaments of small thickness. The thickness of these film-shaped medicaments is 0.01 mm to 5 mm, preferably 0.03 to 3 mm, especially preferably 0.05 mm to 2 mm, and with even greater preference 0.1 mm to 1 mm. The area of the inventive medicaments is between 0.5 to 20 cm²; preferably, the area amounts to 1 to 10 cm².

The shape of the individual medicaments may vary. They may be of round, oval, triangular, rectangular or polygonal shape.

The medicaments according to the invention are also called “wafers”. They are capable of conforming to the irregular surface contour of the oral mucosa after having absorbed moisture. In addition, the medicaments according to the invention may be gelatinizable or capable of swelling.

In a preferred embodiment, the inventive film-shaped medicinal preparations are already pliable before they are applied and can absorb moisture from the saliva.

The active substance content of a film-shaped medicinal preparation according to the invention is 0.5 to 40%-wt, preferably 1 to 30%-wt, and particularly preferably 5 to 20%-wt.

The film-shaped medicaments consist of a polymer-containing layer or comprise at least one polymer-containing layer that serves as an active substance reservoir. This layer contains the active substance and is capable of liberating it upon action of saliva. The polymer portion in this polymer-containing reservoir layer amounts to 10 to 90%-wt, preferably 20 to 70%-wt, and particularly preferably 30 to 60%-wt.

The polymers suitable for the production of the layer serving as active substance reservoir can be selected from the group which consists of polyvinyl alcohols, polyvinyl pyrrolidones, polyvinyl acetates, polyethylene glycols, polyethylene oxide polymers, polyurethanes, polyacrylic acids, polyacrylates, polymethacrylates, poly(methyl vinyl ether-maleic acid anhydrides), starch, starch derivatives, natural gums, alginates, pectins and gelatine, pullulan, gel-forming proteins, chitosan, agar-agar, agarose, carrageenan, xanthan, tragacanth, dextrane and cellulose ethers such as ethyl cellulose, hydroxyethyl cellulose, propyl cellulose, carboxyl methyl cellulose, Na-carboxymethyl cellulose, hydroxypropyl cellulose, hydroxypropyl methyl cellulose, hydroxypropyl ethyl cellulose, cellulose acetate.

The polymers can be used alone or in combination with one another to prepare a medicament according to the invention which has the properties desired, such as adhesion, release or disintegration properties.

In its simplest embodiment the inventive film-shaped medicament consists of a single polymer layer. Other embodiments concern film-shaped medicaments having a bi- or multilayer structure, with at least one of the layers containing active substance. If a plurality of the layers of these embodiments contains the active substance/active substances, these layers may differ in terms of their active substance content and their active substance composition, but also in terms of their polymer composition and thereby their adhesive properties and/or disintegration properties.

The medicaments according to the invention may additionally contain one or more inactive ingredients known to those skilled in the art. These inactive ingredients may be selected from one or more of the following groups: emulsifiers, comprising polyethoxylated sorbitan fatty acid esters, polyethoxylated fatty alcohols and lecithin; plasticizers, comprising polyethylene glycol, glycerol and other polyalcohols, higher alcohols such as dodecanol, undecanol or octanol, sorbitol, mannitol and other sugar alcohols, dexpanthenol and triglycerides; filling agents, comprising high-disperse silicon dioxide, titanium dioxide, zinc oxide, chalk and starch; colourants; sweeteners and flavouring agents; wetting agents; preservatives; pH regulators and antioxidants; disintegration promoters; permeation promoters improving the absorption of estradiol through the mucosa, e.g. fatty acids and fatty acid esters, polyhydric alcohols such as propanediol, tocopherols or etherial oils such as menthol.

The proportion of these inactive ingredients may amount to up to 60%-wt, relative to the total weight of the medicament. Preferably, the portion of inactive ingredients is 5 to 40%-wt. By adding one or more of the above mentioned inactive ingredients, the skilled artisan is able to exert an influence on the chemical and physical properties of the active substance-containing, film-shaped medicament, so that, for example, a desired flexibility, mucoadhesiveness, swellability or disintegratability as well as diffusion properties can be adjusted.

According to a preferred embodiment, the film-shaped medicaments are to enable a longer-lasting active substance release which is retarded in time.

The active substance is preferably released over a period of 4 hours, especially preferably over a period of 6 hours, and with even greater preference over a period of 8 hours. By a pulse-like release of the active substance within 4 hours, 6 hours or 8 hours once daily, it is possible to produce effects in the central nervous system, the vaginal epithelium and the urogenital tissue which are therapeutically desired, without inducing an atrophy in the vagina or in the urogenital tissues, which have the same origin in terms of embryology.

Pulse-like administration could, for example, contribute to an improved utero-/vaginal dissociation. In this respect the medicament according to the invention can improve the tissue specificity of estriol and the estriol ester(s) in the treatment of climacteric complaints.

In a further preferred embodiment of the medicament according to the invention, the active substance(s) is/are released over a period of 24 hours or longer. It is thereby possible to achieve a largely constant blood level that, due to its favourable effect in the bones, is especially suitable for the treatment of osteoporosis.

To achieve the retarded active substance release in bi- or multilayered medicaments, at least one of the active substance-containing polymer layers has a retarded release of active substance.

To achieve a retarded active substance release, the film-shaped medicaments may preferably be formulated as slowly soluble or slowly disintegrating films which will have completely disintegrated or be completely dissolved only after several hours. Preferably they will have completely disintegrated or be completely dissolved only after 4 hours, especially preferably only after 6 hours, and with greater preference only after 8 hours or even only after 24 hours.

According to a preferred embodiment the film-shaped medicaments according to the invention are mucoadhesive. Particular preference is given to an embodiment which only has one mucoadhesive surface. This enables the medicinal preparation to stick to the oral mucosa during the application period, and the active substance(s) can be absorbed directly at the application site via the oral mucosa.

In a particularly preferred embodiment, the mucoadhesive medicament has, on the side opposite to the mucoadhesive surface, a layer which is impermeable to the active substance, so that upon application to the oral mucosa it is possible to achieve a directional active substance release.

The film-shaped medicament for oral administration, containing estriol and/or at least one pharmacologically acceptable ester of estriol, may additionally contain at least one further active agent from the group of the gestagens which upon application of the medicament is likewise administered transmucosally, so that in a hormone replacement therapy using a combination of estriol with at least one gestagen it is only necessary to administer a single medicinal preparation.

The film-shaped medicaments according to the invention can be produced by methods which are in principle known to the skilled artisan, for example by coating an inert support with a liquid mass comprising polymer(s), active agent(s) and optionally inactive ingredient(s) and solvent(s), for example by means of knife coating, spraying or extrusion processes. The thin film layer thus obtained is dried. In the case of multilayer films, one or more coatings can be applied in the same manner to the existing film layer, or be separately prepared and subsequently laminated thereto.

The inventive film-shaped medicaments for oral application, containing estriol and/or at least one pharmaceutically acceptable ester of estriol, alone or in combination with at least one gestagen, can be used for treating climacteric complaints and/or in the course of a hormone replacement therapy.

Advantageously, the hormone replacement therapy or the treatment of climacteric complaints can be carried out using an estriol dose of less than 2 mg/24 hours, preferably using an active substance dose of approximately 200 μg/24 hours, when the medicament according to the present invention is utilised.

Particularly the pulse-like administration of estriol and its pharmacologically acceptable esters once daily over a period of 4 hours, preferably 6 hours, or particularly preferably 8 hours, which is made possible by the medicament according to the present invention, apart from the administration of smaller doses also enables an improved tissue specificity as compared to transdermal administration.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A film-shaped medicament for oral administration, particularly for buccal administration, wherein said medicament contains at least one of estriol and at least one pharmacologically acceptable ester of estriol, alone or in combination with at least one gestagen, as at least one active substance.

2. The medicament according to claim 1, wherein said medicament comprises at least one polymer-containing layer, said at least one polymer-containing layer containing the at least one active substance and serving as an active substance reservoir.

3. The medicament according to claim 2, wherein said medicament comprises a bilayer or multilayer structure, with at least one layer containing said at least one active substance.

4. The medicament according to claim 2, wherein said at least one polymer-containing layer enables a retarded active substance release.

5. The medicament according to claim 4, wherein said medicament releases the at least one active substance in a time-retarded manner.

6. The medicament according to claim 4, wherein said medicament releases the active substance over a period of at least 24 hours.

7. The medicament according to claim 1, wherein said medicament is gelatinizable or swellable in aqueous media.

8. The medicament according to claim 1, wherein said medicament is soluble or slowly disintegratable in aqueous media.

9. The medicament according to claim 8, wherein said medicament is completely disintegrated or is completely dissolved after 4 hours.

10. The medicament according to claim 1, wherein said medicament releases an active substance dose of less than 2 mg per 24 hours.

11. The medicament according to claim 1, wherein said medicament is mucoadhesive or comprises at least one mucoadhesive surface and wherein a layer of said medicament is averted from said at least one mucoadhesive surface.

12. The medicament according to claim 11, wherein the layer averted from the at least one mucoadhesive surface is impermeable to the at least one active substance.

13. The medicament according to claim 2, wherein the polymer portion in the polymer-containing layer is 10 to 90%-wt.

14. The medicament according to claim 1, wherein said medicament comprises an active substance content of 0.5 to 40%-wt.

15. The medicament according to claim 1, wherein the thickness of the medicament is 0.01 mm to 5 mm.

16. Use of estriol and/or at least one of its pharmaceutically acceptable esters for producing a film-shaped medicament for oral administration and for treating climacteric complaints.

17. Use according to claim 16, wherein said film-shaped medicament is a medicament according to claim 1.

18. Use of a medicament according to claim 1 for treating climacteric complaints.

19. The medicament according to claim 5, wherein said medicament releases the at least one active substance over a period of 4 hours.

20. The medicament according to claim 19, wherein said medicament releases said at least one active substance in a time-retarded manner over a period of 6 hours.

21. The medicament according to claim 20, wherein said medicament releases said at least one active substance in a time-retarded manner over a period of 8 hours.

22. The medicament according to claim 9, wherein said medicament is completely disintegrated or is completely dissolved after 6 hours.

23. The medicament according to claim 22, wherein said medicament is completely disintegrated or is completely dissolved after 8 hours.

24. The medicament according to claim 23, wherein said medicament is completely disintegrated or is completely dissolved after 24 hours.

25. The medicament according to claim 10, wherein said medicament releases an active substance dose of approximately 200 μg per 24 hours.

26. The medicament according to claim 13, wherein the polymer portion in the polymer-containing layer is 20 to 70%-wt.

27. The medicament according to claim 26, wherein the polymer portion in the polymer-containing layer is 30 to 60%-wt.

28. The medicament according to claim 14, wherein said medicament comprises an active substance content of 1 to 30%-wt.

29. The medicament according to claim 28 wherein said medicament comprises an active substance content of 5 to 20%-wt.

30. The medicament according to claim 15, wherein the thickness of the medicament is 0.03 to 3 mm.

31. The medicament according to claim 30, wherein the thickness of the medicament is 0.05 to 2 mm.

32. The medicament according to claim 31, wherein the thickness of the medicament is 0.1 to 1 mm.
